(19) United States
(12) Patent Application Publication
(10) Pub. No.: US 2026/0118675 A1
(43) Pub. Date: Apr. 30, 2026

(54) SYSTEMS AND METHODS FOR MULTIDIRECTIONAL SENSORS

(71) Applicant: Apple Inc., Cupertino, CA (US)

(72) Inventors: Thilaka S. SUMANAWEERA, San Jose, CA (US); Seyed Mohammad Amin MOTAHARI BIDGOLI, Shoreline, WA (US); Xinsheng CHU, Saratoga, CA (US); Gopal VALSAN, Gilroy, CA (US)

(21) Appl. No.: 19/003,452

(22) Filed: Dec. 27, 2024

Related U.S. Application Data
(60) Provisional application No. 63/616,492, filed on Dec. 29, 2023.

(51) Int. Cl. G02B 27/01; A61B 5/00; A61B 5/01; A61B 5/08; G01J 5/0806; G02B 27/00

(52) U.S. Cl. CPC G02B 27/0172; A61B 5/01; A61B 5/0803; A61B 5/6803; A61B 5/7235; A61B 5/7278; G01J 5/0806; A61B 2560/0462; A61B 2562/0271; G02B 27/0093; G02B 2027/014

(57) ABSTRACT

A system including a wearable device designed for real-time breath monitoring of a user, featuring multidirectional sensors capable of receiving light signals from subsections of a target area at a user of the wearable device. In some examples, the wearable device includes one or more multidirectional sensors positioned to capture light signals from a first field of view and a second field of view via an integrated lens focusing the received light signals to be detected by one or more pixels of the multidirectional sensor. In some examples, a breathing signal is calculated using processed light signals from the distinct subsections of the user's face.

### CROSS-REFERENCE TO RELATED APPLICATIONS

This application claims benefit of U.S. Provisional Ser. No. 63/616,492, filed Dec. 29, 2023, the contents of all of which are hereby incorporated by reference in their entirety for all purposes.

### FIELD OF THE DISCLOSURE

This disclosure relates generally to an electronic device incorporating a multidirectional sensor, and more particularly, to an electronic device incorporating a multidirectional thermal sensor.

### BACKGROUND OF THE DISCLOSURE

Aspects of this disclosure relate to non-invasive temperature sensing methods. Common strategies employing multiple thermal sensors in remote sensing technologies, while effective, often introduce mechanical complexity and increased costs.

### SUMMARY OF THE DISCLOSURE

This disclosure relates generally to an electronic device incorporating a multidirectional sensor, and more particularly, to an electronic device incorporating a multidirectional thermal sensor. In some examples, a wearable device, such as a head-mounted display, includes a multidirectional thermal sensor. The wearable device optionally uses time-division multiplexing, a dual lens/shutter system, and/or a waveguide to achieve multiple fields of view. For example, the multidirectional thermal sensor optionally incorporates pixel activation/deactivation at the sensor in accordance with time-division multiplexing to achieve the multiple fields of view and increase the precision of the sensor. In some examples, the multidirectional thermal sensor is configured to scan the face of the user wearing the wearable device to sense temperature changes induced by breathing. In some examples, the wearable device uses the collected data to generate a breathing signal associated with the user. This approach enhances applications in health monitoring and biometrics, providing non-intrusive and continuous insights into the user's well-being.

### BRIEF DESCRIPTION OF THE DRAWINGS

For a better understanding of the various described examples, reference should be made to the Detailed Description below, in conjunction with the following drawings in which like reference numerals refer to the corresponding parts throughout the figures.

FIG. 1A illustrates a front, top, perspective view of an example of a head-mountable display device (herein after referred to as a wearable device) configured to be donned by a user and provide virtual and augmented/mixed reality (VR/AR) experiences according to some examples of the disclosure.

FIG. 1B illustrates a rear, perspective view of the wearable device according to some examples of the disclosure.

FIG. 2 is a block diagram of an example electronic device, such as a wearable device, according to some examples of the disclosure.

FIGS. 3A-3D illustrate example configurations of sensors, including multidirectional sensors, affixed to a wearable device that can be configured to measure a plurality of light signals emanating from a user according to some examples of the disclosure.

FIG. 3E illustrates an example of the components of the multidirectional sensors described above with reference to FIGS. 3A-3D according to some examples of the disclosure.

FIGS. 4A-4H illustrate example configurations of photodetectors sampling a plurality of light signals from multiple directions according to some examples of this disclosure.

FIGS. 5A-5E illustrate examples of an array of pixels at a multidirectional sensor operating in a time-division multiplexed manner to generate a thermal image of a user in accordance with some examples of this disclosure.

FIG. 6 illustrates an example of wearable device including a multidirectional sensor 602 that includes an optical waveguide in accordance with some examples of the disclosure.

FIG. 7 is a flow diagram illustrating a method of generating a breathing signal associated with the user according to some examples of this disclosure.

### DETAILED DESCRIPTION

The present disclosure relates to various examples for providing remote thermal measurements of a user using a wearable device, in accordance with some examples. In the following description of examples, reference is made to the accompanying drawings which form a part hereof, and in which it is shown by way of illustration specific examples that can be practiced. It is to be understood that other examples can be used and structural changes can be made without departing from the scope of the disclosed examples.

This disclosure relates generally to an electronic device incorporating a multidirectional sensor, and more particularly, to an electronic device incorporating a multidirectional thermal sensor. In some examples, a wearable device, such as a head-mounted display, includes a multidirectional thermal sensor. The wearable device optionally uses time-division multiplexing, a dual lens/shutter system, and/or a waveguide to achieve multiple fields of view. For example, the multidirectional thermal sensor optionally incorporates pixel activation/deactivation at the sensor in accordance with time-division multiplexing to achieve the multiple fields of view and increase the precision of the sensor. In some examples, the multidirectional thermal sensor is configured to scan the face of the user wearing the wearable device to sense temperature changes induced by breathing. In some examples, the wearable device uses the collected data to generate a breathing signal associated with the user. This approach enhances applications in health monitoring and biometrics, providing non-intrusive and continuous insights into the user's well-being.

The methods and devices described herein improve thermal measurements in multiple ways for the purpose of improving the detection of breathing of a user of the device. In some examples, the device uses sensors to collect data including physiological data of the user of the device including tissue temperature of the user at different locations and/or along different directions. In some examples, the device uses senses a respiratory surrogate waveform, induced by the user breathing, by sensing temperature fluctuations at the nose and the mouth of the user (herein after referred to as a breathing signal). In some examples, in response to sensing the breathing signal, the device will generate an alert to regulate breathing. For example, the alert includes causing the device to output a visual, audio and/or tactile indication to the user and/or altering a device used by another user, such as a partner, exercise coach, and/or a user in wireless communication with the user of the device.

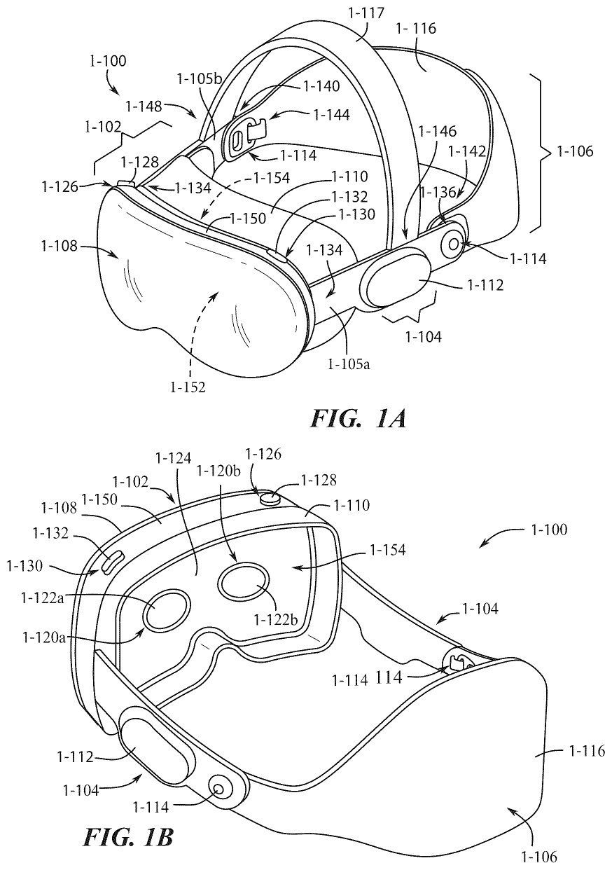

FIG. 1A illustrates a front, top, perspective view of an example of a head-mountable display device 1-100 (herein after referred to as a wearable device) configured to be donned by a user and provide virtual and altered/mixed reality (VR/AR) experiences according to some examples of the disclosure. The wearable device 1-100 can include a display unit 1-102 or assembly, an electronic strap assembly 1-104 connected to and extending from the display unit 1-102, and a band assembly 1-106 secured at either end to the electronic strap assembly 1-104. The electronic strap assembly 1-104 and the band 1-106 can be part of a retention assembly configured to wrap around a user's head to hold the display unit 1-102 against the face of the user.

In at least one example, the band assembly 1-106 can include a first band 1-116 configured to wrap around the rear side of a user's head and a second band 1-117 configured to extend over the top of a user's head. The second strap can extend between first and second electronic straps 1-105a, 1-105b of the electronic strap assembly 1-104 as shown. The strap assembly 1-104 and the band assembly 1-106 can be part of a securement mechanism extending rearward from the display unit 1-102 and configured to hold the display unit 1-102 against a face of a user.

In at least one example, the securement mechanism includes a first electronic strap 1-105a including a first proximal end 1-134 coupled to the display unit 1-102, for example a housing 1-150 of the display unit 1-102, and a first distal end 1-136 opposite the first proximal end 1-134. The securement mechanism can also include a second electronic strap 1-105b including a second proximal end 1-138 coupled to the housing 1-150 of the display unit 1-102 and a second distal end 1-140 opposite the second proximal end 1-138. The securement mechanism can also include the first band 1-116 including a first end 1-142 coupled to the first distal end 1-136 and a second end 1-144 coupled to the second distal end 1-140 and the second band 1-117 extending between the first electronic strap 1-105a and the second electronic strap 1-105b. The straps 1-105a-b and band 1-116 can be coupled via connection mechanisms or assemblies 1-114. In at least one example, the second band 1-117 includes a first end 1-146 coupled to the first electronic strap 1-105a between the first proximal end 1-134 and the first distal end 1-136 and a second end 1-148 coupled to the second electronic strap 1-105b between the second proximal end 1-138 and the second distal end 1-140.

In at least one example, the first and second electronic straps 1-105a-b include plastic, metal, or other structural materials forming the shape the substantially rigid straps 1-105a-b. In at least one example, the first and second bands 1-116, 1-117 are formed of elastic, flexible materials including woven textiles, rubbers, and the like. The first and second bands 1-116, 1-117 can be flexible to conform to the shape of the user′ head when donning the wearable device 1-100.

In at least one example, one or more of the first and second electronic straps 1-105a-b can define internal strap volumes and include one or more electronic components disposed in the internal strap volumes. In one example, as shown in FIG. 1A, the first electronic strap 1-105a can include an electronic component 1-112. In one example, the electronic component 1-112 can include a speaker. In one example, the electronic component 1-112 can include a computing component such as a processor.

In at least one example, the housing 1-150 defines a first, front-facing opening 1-152. The front-facing opening is labeled in dotted lines at 1-152 in FIG. 1B because the display assembly 1-108 is disposed to occlude the first opening 1-152 from view when the wearable device 1-100 is assembled. The housing 1-150 can also define a rear-facing second opening 1-154. The housing 1-150 also defines an internal volume between the first and second openings 1-152, 1-154. In at least one example, the wearable device 1-100 includes the display assembly 1-108, which can include a front cover and display screen (shown in other figures) disposed in or across the front opening 1-152 to occlude the front opening 1-152. In at least one example, the display screen of the display assembly 1-108, as well as the display assembly 1-108 in general, has a curvature configured to follow the curvature of a user's face. The display screen of the display assembly 1-108 can be curved as shown to compliment the user's facial features and general curvature from one side of the face to the other, for example from left to right and/or from top to bottom where the display unit 1-102 is pressed.

In at least one example, the housing 1-150 can define a first aperture 1-126 between the first and second openings 1-152, 1-154 and a second aperture 1-130 between the first and second openings 1-152, 1-154. The wearable device 1-100 can also include a first button 1-128 disposed in the first aperture 1-126 and a second button 1-132 disposed in the second aperture 1-130. The first and second buttons 1-128, 1-132 can be depressible through the respective apertures 1-126, 1-130. In at least one example, the first button 1-126 and/or second button 1-132 can be twistable dials as well as depressible buttons. In at least one example, the first button 1-128 is a depressible and twistable dial button and the second button 1-132 is a depressible button.

FIG. 1B illustrates a rear, perspective view of the wearable device 1-100 according to some examples of the disclosure. The wearable device 1-100 can include a light seal 1-110 extending rearward from the housing 1-150 of the display assembly 1-108 around a perimeter of the housing 1-150 as shown. The light seal 1-110 can be configured to extend from the housing 1-150 to the user's face around the user's eyes to block external light from being visible. In one example, the wearable device 1-100 can include first and second display assemblies 1-120a, 1-120b disposed at or in the rearward facing second opening 1-154 defined by the housing 1-150 and/or disposed in the internal volume of the housing 1-150 and configured to project light through the second opening 1-154. In at least one example, each display assembly 1-120a-b can include respective display screens 1-122a, 1-122b configured to project light in a rearward direction through the second opening 1-154 toward the user's eyes.

In at least one example, referring to both FIGS. 1A and 1B, the display assembly 1-108 can be a front-facing, forward display assembly including a display screen configured to project light in a first, forward direction and the rear facing display screens 1-122a-b can be configured to project light in a second, rearward direction opposite the first direction. As noted above, the light seal 1-110 can be configured to block light external to the wearable device 1-100 from reaching the user's eyes, including light projected by the forward-facing display screen of the display assembly 1-108 shown in the front perspective view of FIG. 1B. In at least one example, the wearable device 1-100 can also include a curtain 1-124 occluding the second opening 1-154 between the housing 1-150 and the rear-facing display assemblies 1-120a-b. In at least one example, the curtain 1-124 can be elastic or at least partially elastic.

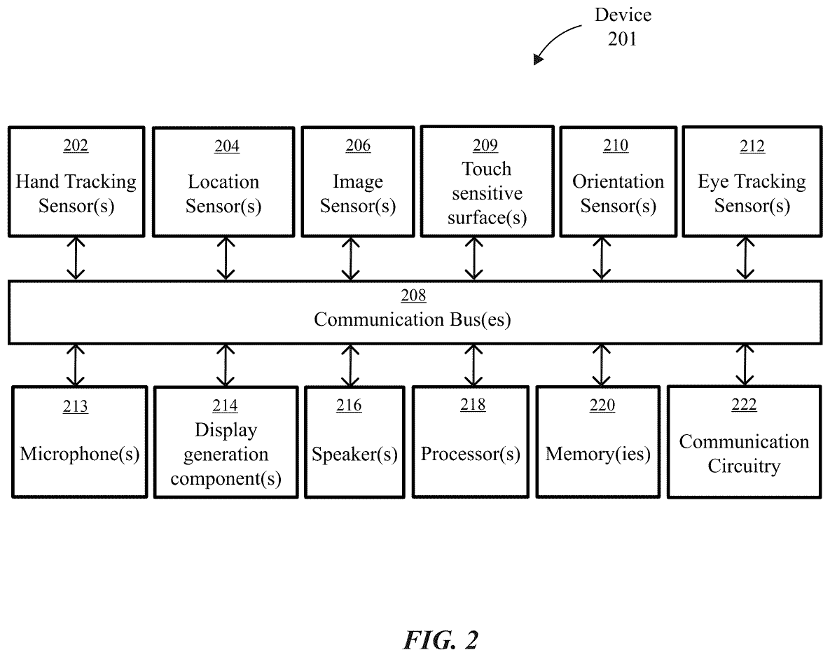

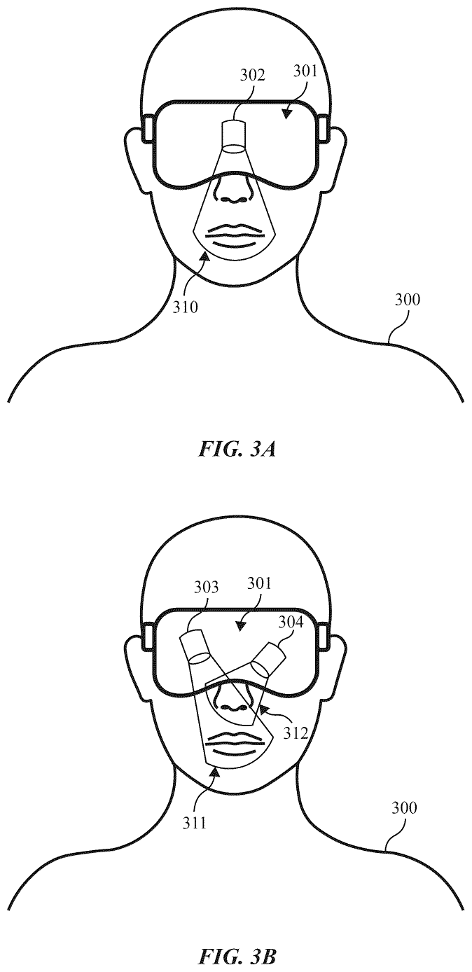

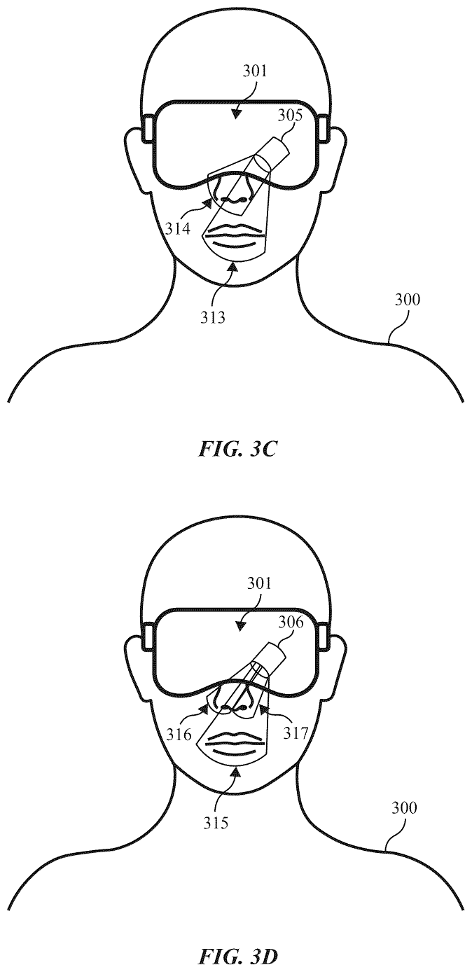

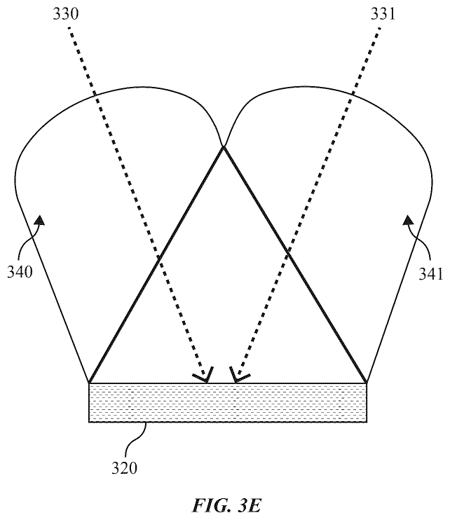

FIG. 2 is a block diagram of an example electronic device, such as a wearable device, according to some examples of the disclosure. In some examples, as illustrated in FIG. 2, the electronic device 201 includes various sensors, such as one or more hand tracking sensors 202, one or more location sensors 204, one or more image sensors 206, one or more touch-sensitive surfaces 209, one or more motion and/or orientation sensors 210, one or more eye tracking sensors 212, one or more microphones 213 or other audio sensors, one or more body tracking sensors (e.g., torso and/or head tracking sensors), one or more display generation components 214, optionally corresponding to displays 1-122a and 1-122b in FIG. 1, one or more speakers 216, one or more processors 218, one or more memories 220, and/or communication circuitry 222. Additionally or alternatively, in some examples, the electronic device 201 further includes a multidirectional sensor, such as a multidirectional thermal sensor, according to one or more examples described with reference to FIGS. 3A-7 below. One or more communication buses 208 are optionally used for communication between the above-mentioned components of electronic devices 201. In some examples, the electronic device 201 optionally corresponds to the wearable device 1-100 illustrated in FIGS. 1A and 1B.

Communication circuitry 222 optionally includes circuitry for communicating with electronic devices, networks, such as the Internet, intranets, a wired network and/or a wireless network, cellular networks, and wireless local area networks (LANs). Communication circuitry 222 optionally includes circuitry for communicating using near-field communication (NFC) and/or short-range communication, such as Bluetooth®.

Processor(s) 218 include one or more general processors, one or more graphics processors, and/or one or more digital signal processors. In some examples, memory 220 is a non-transitory computer-readable storage medium (e.g., flash memory, random access memory, or other volatile or non-volatile memory or storage) that stores computer-readable instructions configured to be executed by processor(s) 218 to perform the techniques, processes, and/or methods described below. In some examples, memory 220 can include more than one non-transitory computer-readable storage medium. A non-transitory computer-readable storage medium can be any medium (e.g., excluding a signal) that can tangibly contain or store computer-executable instructions for use by or in connection with the instruction execution system, apparatus, or device. In some examples, the storage medium is a transitory computer-readable storage medium. In some examples, the storage medium is a non-transitory computer-readable storage medium. The non-transitory computer-readable storage medium can include, but is not limited to, magnetic, optical, and/or semiconductor storages. Examples of such storage include magnetic disks, optical discs based on compact disc (CD), digital versatile disc (DVD), or Blu-ray technologies, as well as persistent solid-state memory such as flash, solid-state drives, and the like.

In some examples, display generation component(s) 214 include a single display (e.g., a liquid-crystal display (LCD), organic light-emitting diode (OLED), or other types of display). In some examples, display generation component(s) 214 includes multiple displays. In some examples, display generation component(s) 214 can include a display with touch capability (e.g., a touch screen), a projector, a holographic projector, a retinal projector, a transparent or translucent display, etc. In some examples, electronic device 201 includes touch-sensitive surface(s) 209, respectively, for receiving user inputs, such as tap inputs and swipe inputs or other gestures. In some examples, display generation component(s) 214 and touch-sensitive surface(s) 209 form touch-sensitive display(s) (e.g., a touch screen integrated with electronic device 201 or external to electronic device 201 that is in communication with electronic device 201).

Electronic device 201 optionally includes image sensor(s) 206. Image sensors(s) 206 optionally include one or more visible light image sensors, such as charged coupled device (CCD) sensors, and/or complementary metal-oxide-semiconductor (CMOS) sensors operable to obtain images of physical objects from the real-world environment. Image sensor(s) 206 also optionally include one or more infrared (IR) sensors, such as a passive or an active IR sensor, for detecting infrared light from the real-world environment. For example, an active IR sensor includes an IR emitter for emitting infrared light into the real-world environment. Image sensor(s) 206 also optionally include one or more cameras configured to capture movement of physical objects in the real-world environment. Image sensor(s) 206 also optionally include one or more depth sensors configured to detect the distance of physical objects from electronic device 201. In some examples, information from one or more depth sensors can allow the device to identify and differentiate objects in the real-world environment from other objects in the real-world environment. In some examples, one or more depth sensors can allow the device to determine the texture and/or topography of objects in the real-world environment.

In some examples, electronic device 201 uses CCD sensors, event cameras, and depth sensors in combination to detect the physical environment around electronic device 201. In some examples, image sensor(s) 206 include a first image sensor and a second image sensor. The first image sensor and the second image sensor work in tandem and are optionally configured to capture different information of physical objects in the real-world environment. In some examples, the first image sensor is a visible light image sensor and the second image sensor is a depth sensor. In some examples, electronic device 201 uses image sensor(s) 206 to detect the position and orientation of electronic device 201 and/or display generation component(s) 214 in the real-world environment. For example, electronic device 201 uses image sensor(s) 206 to track the position and orientation of display generation component(s) 214 relative to one or more fixed objects in the real-world environment.

In some examples, electronic device 201 includes microphone(s) 213 or other audio sensors. Electronic device 201 optionally uses microphone(s) 213 to detect sound from the user and/or the real-world environment of the user. In some examples, microphone(s) 213 includes an array of microphones (a plurality of microphones) that optionally operate in tandem, such as to identify ambient noise or to locate the source of sound in space of the real-world environment.

Electronic device 201 includes location sensor(s) 204 for detecting a location of electronic device 201 and/or display generation component(s) 214. For example, location sensor(s) 204 can include a global positioning system (GPS) receiver or similar that receives data from one or more satellites and allows electronic device 201 to determine the device's absolute position in the physical world.

Electronic device 201 includes orientation sensor(s) 210 for detecting orientation and/or movement of electronic device 201 and/or display generation component(s) 214. For example, electronic device 201 uses orientation sensor(s) 210 to track changes in the position and/or orientation of electronic device 201 and/or display generation component(s) 214, such as with respect to physical objects in the real-world environment. Orientation sensor(s) 210 optionally include one or more gyroscopes and/or one or more accelerometers.

Electronic device 201 includes hand tracking sensor(s) 202 and/or eye tracking sensor(s) 212 (and/or other body tracking sensor(s), such as leg, torso and/or head tracking sensor(s)), in some examples. Hand tracking sensor(s) 202 are configured to track the position/location of one or more portions of the user's hands, and/or motions of one or more portions of the user's hands with respect to the extended reality environment, relative to the display generation component(s) 214, and/or relative to another defined coordinate system. Eye tracking sensor(s) 212 are configured to track the position and movement of a user's gaze (eyes, face, or head, more generally) with respect to the real-world or extended reality environment and/or relative to the display generation component(s) 214. In some examples, hand tracking sensor(s) 202 and/or eye tracking sensor(s) 212 are implemented together with the display generation component(s) 214. In some examples, the hand tracking sensor(s) 202 and/or eye tracking sensor(s) 212 are implemented separate from the display generation component(s) 214.

In some examples, the hand tracking sensor(s) 202 (and/or other body tracking sensor(s), such as leg, torso and/or head tracking sensor(s)) can use image sensor(s) 206 (e.g., one or more IR cameras, 3D cameras, depth cameras, etc.) that capture three-dimensional information from the real-world including one or more body parts (e.g., hands, legs, or torso of a human user). In some examples, the hands can be resolved with sufficient resolution to distinguish fingers and their respective positions. In some examples, one or more image sensors 206 are positioned relative to the user to define a field of view of the image sensor(s) 206 and an interaction space in which finger/hand position, orientation and/or movement captured by the image sensors are used as inputs (e.g., to distinguish from a user's resting hand or other hands of other persons in the real-world environment). Tracking the fingers/hands for input (e.g., gestures, touch, tap, etc.) can be advantageous in that it does not require the user to touch, hold or wear any sort of beacon, sensor, or other marker.

In some examples, eye tracking sensor(s) 212 includes at least one eye tracking camera (e.g., infrared (IR) cameras) and/or illumination sources (e.g., IR light sources, such as LEDs) that emit light towards a user's eyes. The eye tracking cameras may be pointed towards a user's eyes to receive reflected IR light from the light sources directly or indirectly from the eyes. In some examples, both eyes are tracked separately by respective eye tracking cameras and illumination sources, and a focus/gaze can be determined from tracking both eyes. In some examples, one eye (e.g., a dominant eye) is tracked by one or more respective eye tracking cameras/illumination sources.

Electronic device 201 is not limited to the components and configuration of FIG. 2, but can include fewer, other, or additional components in multiple configurations. In some examples, electronic device 201 can be implemented between two electronic devices (e.g., as a system). In some such examples, each of (or more) electronic device may each include one or more of the same components discussed above, such as various sensors, one or more display generation components, one or more speakers, one or more processors, one or more memories, and/or communication circuitry. A person or persons using electronic device 201, is optionally referred to herein as a user or users of the device.

FIGS. 3A-3D illustrate example configurations of sensors 302, 303, 304, 305, and 306, including multidirectional sensors 305 and 306, affixed to a wearable device 301 that can be configured to measure a plurality of light signals corresponding to photons generated by a user 300 according to some examples of the disclosure. In some examples, the wearable device 301 is configured to calculate a breathing signal associated with the user 300 as described herein (e.g., using the plurality of light signals gathered within a field of view of the multidirectional sensors 302-306 described in further detail below). In some examples, the multidirectional sensors 305 and 306 include a dual-lens component as illustrated in FIG. 3E described in further detail below. Additional or alternative components can be included in the wearable device 301 without departing from the scope of the disclosure.

FIG. 3A illustrates an example of the wearable device 301 being worn by the user 300 according to some examples of the disclosure. In some examples, the wearable device 301 corresponds to the head-mountable display device 1-100 described above with reference to FIGS. 1A and 1B. The wearable device 301 can include a strap affixed around the temples of the user 300 head, as shown in FIG. 3A, allowing the user 300 to view the interior of the wearable device that, in some examples, corresponds to the display unit 1-102 described above with reference to FIGS. 1A and 1B. In some examples, the wearable device 301 includes the sensor 302 disposed within or outside the surface of the wearable device 301. The sensor 302 includes a field of view 310 that can be orientated to several subsections of a target area of the user 300 that encompass both the nose and mouth of the user 300. For example, the field of view 310 corresponds to two subsections of the target area of the user 300 (e.g., including both the nose and the mouth of the user 300) where the nose corresponds to a first subsection of the target area of the user and the mouth corresponds to a second subsection of the target area of the user. In some examples, the field of view 310 corresponds to the sensing range of the sensor 302. In some examples, the plurality of light signals corresponds to light signals within the field of view 310. In some examples, the plurality of light signals are generated by changes in temperature at user 300 induced by breathing at user 300. For example, the user can exhale through the mouth and/or nose, as illustrated in FIG. 3A, triggering a change in thermal energy within the field of view 310. This change in thermal energy at the user 300 mouth and/or nose emits the plurality of light signals within the field of view 310 that are subsequently captured by the sensor 302. Additionally, or alternatively, the plurality of light signals corresponds to photons with a wavelength greater than the visible light spectrum (i.e., a wavelength larger than 780 nanometers). For example, the plurality of light signals generated by user 300 can possess a wavelength within a range of 780 nanometers to 1 millimeter corresponding to photons within the infrared spectrum. In some examples, sensor 302 includes a focusing element (e.g., an optical lens) that determines the field of view 310. For example, field of view 310 is configured to encompass the target area of user 300 mouth (e.g., nose and mouth) optionally without encompassing other areas. In view of this restriction, and assuming an average vertical distance between the nose and mouth of user 300 to be between 11-13 mm, sensor 302 optionally includes an optical lens with a focal length between 5-10 mm that results in the field of view 310 between 10-15 mm. In restricting the field of view 310 to encompass the nose and mouth of the user 300 optionally without encompassing other areas, extraneous sources of light signals are reduced, resulting in an increase in the accuracy of the detection of the plurality of light signals.

FIG. 3B illustrates an example configuration of the wearable device 301 that includes sensors 303 and 304 affixed to the wearable device 301 according to some examples of the disclosure. In some examples, sensor 303 includes a field of view 311 that corresponds to the sensing range of the sensor 303. For example, while the user 300 is breathing, the sensor 303 detects the plurality of light signals at the mouth of the user 300 within the field of view 311. In addition, while the user 300 is breathing, sensor 304 detects the plurality of light signals at the user 300 nose within the field of view 312, where the field of view 312 corresponds to the sensing range of sensor 304. In some examples, the use of one sensor 303 (e.g., without an additional sensor of the same type, such as sensor 304) to detect the plurality of light signals results in reduced cost, reduced mechanical complexity of the wearable device 301 and other advantages discussed in further detail below.

FIG. 3C illustrates an example configuration of the wearable device 301 that includes a multidirectional sensor 305 affixed to the wearable device 301 according to some examples of the disclosure. In some examples, the multidirectional sensor 305 includes a dual sensing range corresponding to the field of view 313 and the field of view 314. In some examples, the field of view 313 and the field of view 314 are time-division multiplexed, as described in further detail below. For example, the multidirectional sensor 305 may detect the plurality of light signals within the field of view 313 for a predetermined time (e.g., 5, 10, 15, or 30 milliseconds) before alternating to sensing the plurality of light signals within the field of view 314 for the predetermined time. Additionally or alternatively, the predetermined time associated with field of view 313 does not equal the predetermined time associated with field of view 314. In some examples, the field of view 313 encompasses the mouth of the user 300 and corresponds to the emission of the plurality of light signals triggered by the change in temperature at the user 300 mouth. In some examples, the field of view 314 encompasses the nose of the user 300 and corresponds to the emission of the plurality of light signals triggered by the change in temperature at the user 300 nose. In combination with the above-described detected plurality of light signals within the field of view 313 and the field of view 314, according to some examples, the detected plurality of light signals are absorbed and converted into a plurality of electrical signals via one or more pixels at the multidirectional sensor 305 (e.g., photodiodes, avalanche photodiodes, balanced detectors, photomultiplier tubes, etc.) wherein processing circuitry at the multidirectional sensor 305 processes the plurality of electrical signals and generates the breathing signal associated with the user 300. In some examples, the multidirectional sensor 305 uses one or more pixels of one or more combinations of the above-mentioned examples of pixels.

FIG. 3D illustrates an example configuration of the wearable device 301 that includes a multidirectional sensor 306 affixed to the wearable device 301 according to some examples of the disclosure. In some examples, the multidirectional sensor 306 includes a triple sensing range corresponding to the field of view 315, the field of view 316, and the field of view 317. In some examples, the field of view 315, the field of view 316, and the field of view 317 are time-division multiplexed in a similar fashion as described above with reference to FIG. 3C. In some examples, the field of view 315 corresponds to a first sensing range of the multidirectional sensor 306 wherein the plurality of light signals emitted from the mouth of the user 300 are detected. In some examples, the field of view 316 corresponds to a second sensing range of the multidirectional sensor 306 wherein the plurality of light signals emitted from a first nostril of the nostrils of the user 300 are detected. In some examples, the field of view 317 corresponds to a third sensing range of the multidirectional sensor 306 wherein the plurality of light signals emitted from a second nostril of the nostrils of the user 300 are detected. As illustrated in FIG. 3D, the first nostril corresponds to the right nostril of the user 300 and the second nostril corresponds to the left nostril of the user 300. Alternatively or additionally, the first nostril corresponds to the left nostril of the user 300 and the second nostril corresponds to the right nostril of the user 300 or any other combination. In some examples, the plurality of light signals collected within the fields of view 315 through 317 are processed at the multidirectional sensor 306 to generate the breathing signal associated with the user 300.

FIG. 3E illustrates an example framework of a base-level configuration of the components of the multidirectional sensors 305 or 306 described above with reference to FIGS. 3A-3D according to some examples of the disclosure. For example, a lens 340 and a lens 341 correspond to the dual-lens configuration of the multidirectional sensor 305 above with reference to FIG. 3C. In some examples, the lens 340 and the lens 341 each corresponds to a respective field of view of the multidirectional sensors 305 or 306 described above with reference to FIGS. 3C-3D. In some examples, one or more pixels 320 are disposed beneath the lens 340 and the lens 341 and are additionally configured to detect the plurality of light signals. For example, the one or more pixels 320 are photodetectors corresponding to the one or more pixels at the multidirectional sensor 305 discussed above with reference to FIG. 3C. In some examples, the plurality of light signals can include a plurality of light signals 330 in a first direction and a plurality of light signals 331 in a second direction. In some examples, the plurality of light signals 330 corresponds to the field of view 314 and the plurality of light signals 331 corresponds to the field of view 315 described above with reference to FIG. 3C. For example, as described above, the plurality of light signals 330 correspond to changes in temperature at the user 300 nostrils and the plurality of light signals 331 correspond to changes in temperature at the user 300 mouth.

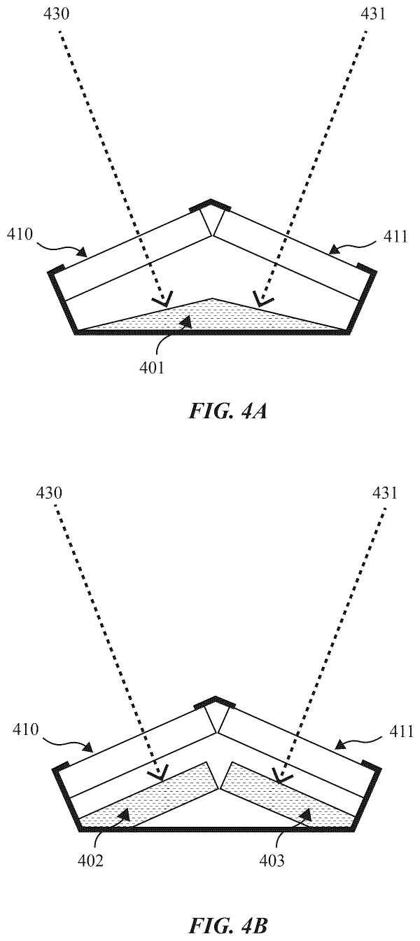

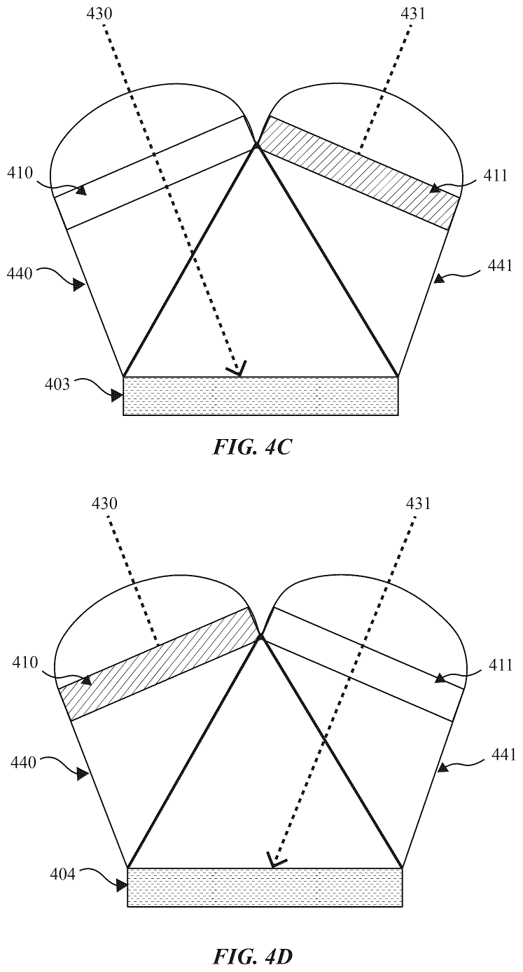

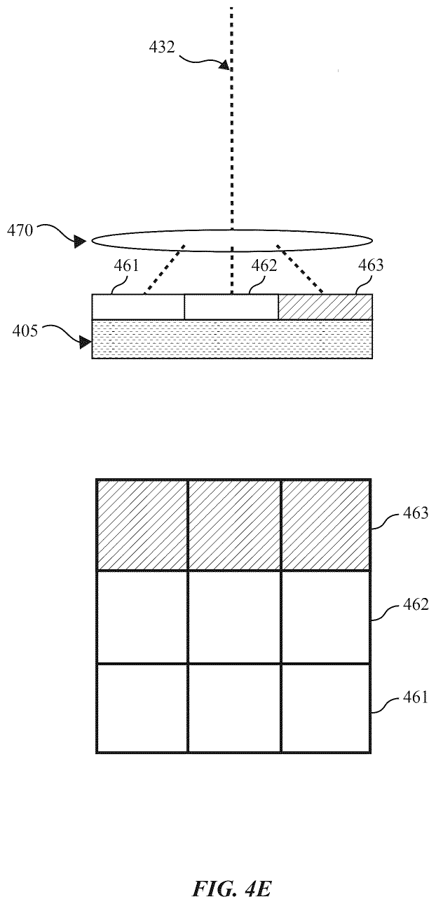

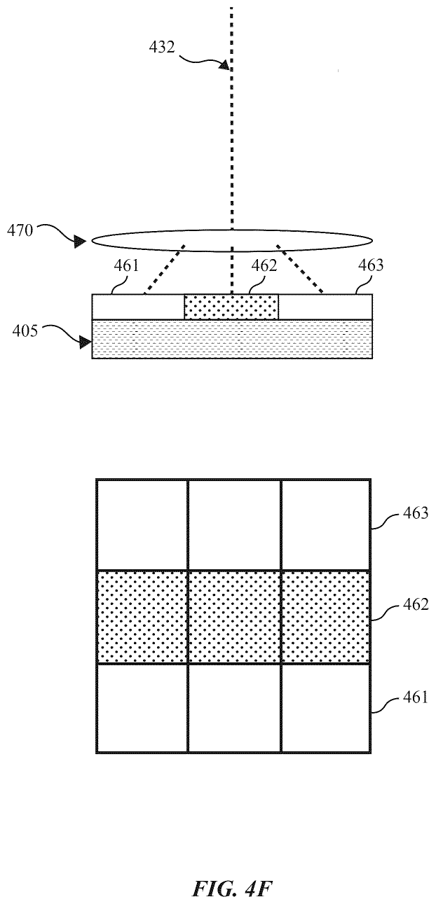

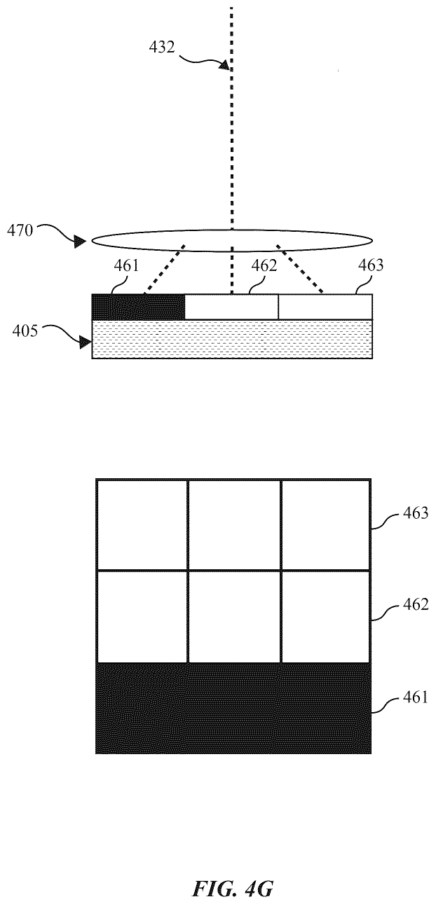

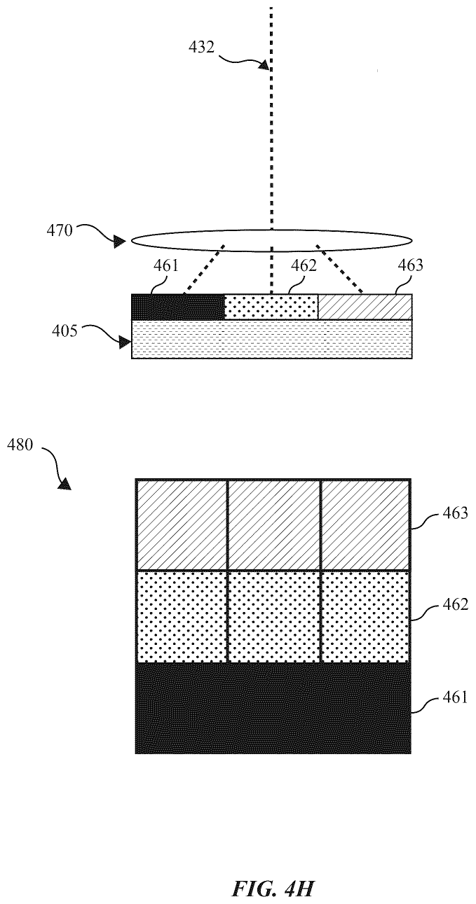

FIGS. 4A-4H illustrate example configurations of photodetectors 401 through 405 sampling a plurality of light signals from multiple directions according to some examples of this disclosure. In some examples these multiple directions correspond to the plurality of light signals 330 and 331 described above with reference to FIG. 3C. In some examples, photodetectors 401 through 405 sample the plurality of light signals from multiple directions according to a plurality of shutters forming an M×N matrix of arbitrary size. In some examples, the multidirectional sensor includes a first shutter 410 and a second shutter 411 that controls the transmission of the plurality of light signals from multiple directions received at the photodetectors 401 through 405. In some examples, shutter rows 461 through 463 form a 3×3 shutter matrix that controls the transmission of the plurality of light signals from multiple directions received at the photodetectors 401 through 405. In this example, (as later shown in FIG. 4H) the 3×3 shutter matrix can generate an image with varying degrees of opacity. In some examples, as illustrated in FIGS. 4C-4D, the wearable device 301 alternates sensing the plurality of light signals from multiple directions through the first shutter and the second shutter to the photodetector 404. In some examples, the photodetector 404 is a two-dimensional array of pixels that are configured by the shutter system to activate and/or deactivate in a predetermined pattern. In some examples, the plurality of light signals from the multiple directions are sampled at the photodetector 404 within the multidirectional sensor in a time-division multiplex fashion according to some examples of the disclosure. In some examples, the photodetector 404 is one of multidirectional sensors 302 through 306 included in FIGS. 3A-3E.

FIG. 4A illustrates an example configuration of a shutter system including the first shutter 410 and the second shutter 411 fixed above the photodetector 401 within a multidirectional sensor according to some examples of this disclosure. In some examples the multidirectional sensor includes a triangle shape, thus allowing the photodetector 401 to receive a plurality of light signals 430 from a first direction, and a plurality of light signals 431 from a second direction. In some examples, other shapes that enable the photodetector 401 to receive light signals from a different number of directions are possible. In some examples, as illustrated in FIG. 4A, the multidirectional sensor comprises a single pixel configured to detect the plurality of light signals 430 from the first direction and the plurality of light signals 431 from the second direction via the triangle shape possessing a first and second side facing the first shutter 410 and the second shutter 411, respectively. In some examples, the first shutter 410 and the second shutter 411 include silicon windows, wherein each shutter acts as a focusing element to focus the plurality of light signals from a respective direction. For example, the first shutter 410 focuses the plurality of light signals 430 from the first direction onto the face of the first side of the photodetector 401 while the second shutter 411 focuses the plurality of light signals 431 from the second direction onto the face of the second side of the photodetector 401. In some examples, the photodetector 401 is a single pixel corresponding to the example types of photodetectors discussed above with relation to FIG. 3C. In some examples, the plurality of light signals 430 from the first direction and the plurality of light signals 431 from the second direction are summed by processing circuity operatively coupled to the multidirectional sensors encompassing the photodetector 401. In some examples, the first shutter 410 and the second shutter 411 alter their respective opacities to reduce or prevent the transmission of the plurality of light signals 430 and 431 to the photodetector 401 discussed in further detail below. In some examples, the plurality of light signals 430 from the first direction and the plurality of light signals 431 from the second direction correspond to the plurality of light signals 330 and the plurality of light signals 331, respectively, as discussed above with reference to FIG. 3E.

FIG. 4B illustrates an example configuration of the first shutter 410 and the second shutter 411 affixed above a dual-photodetector (photodetectors 402 and 403 discussed in further detail below) as discussed above with reference to FIG. 4A including a multiple pixel configuration according to some examples of this disclosure. In this example, as illustrated by FIG. 4B, the dual-photodetector comprises a first photodetector 402 and a second photodetector 403 in lieu of the single photodetector illustrated by the photodetector 401 of FIG. 4A. In some examples, the first photodetector 402 receives the plurality of light signals 430 from the first direction and the second photodetector 403 receives the plurality of light signals 431 from the second direction. In some examples, the plurality of light signals received by the first photodetector 402 and the second photodetector 403 are received in a time-division multiplex fashion according to a sampling rate. For example, the sampling rate of the first photodetector 402 and the second photodetector 403 are 10 kHz; therefore, while under time-division multiplexing, the plurality of light signals 430 and 431 are sampled at the first photodetector 402 and the second photodetector 403. In some examples, the first photodetector 402 and the second photodetector 403 detect the plurality of light signals 430 and 431 over the predetermined time similar to the manner described above with reference to FIG. 3C. In some examples, after sensing the plurality of light signals, the wearable device 301 (e.g., the wearable device 301 with reference to FIGS. 3A-3E) generates the breathing signal associated with the user 300 (see FIGS. 3A-3E) using the time-division multiplexed plurality of light signals 430 and 431.

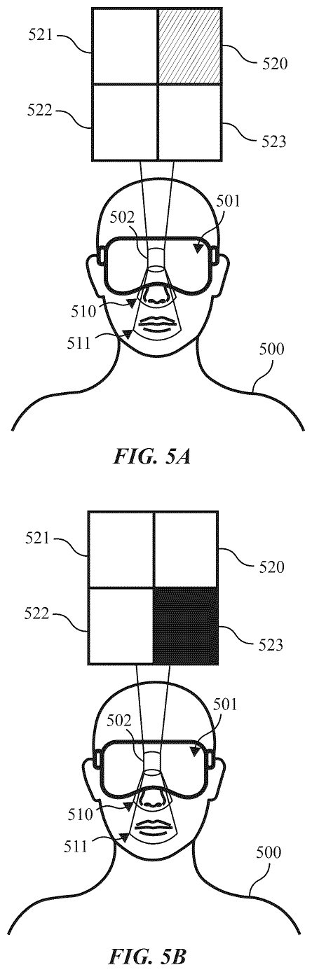

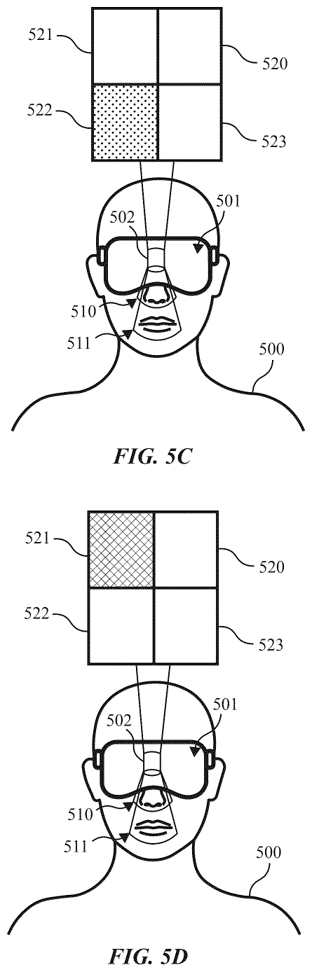

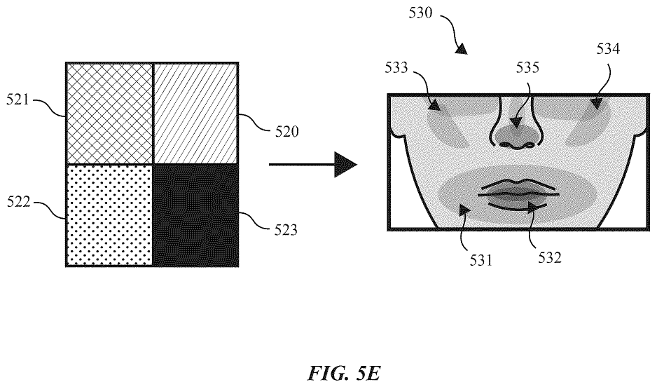

FIG. 4C illustrates a first lens 440 and a second lens 441 in a dual-lens configuration possessing the first shutter 410 and the second shutter 411, disposed within the interior volume of a first lens 440 and a second lens 441 respectively, in a first stage of time-division multiplexing according to some examples of the disclosure. In some examples, the first shutter 410 and the second shutter 411 are disposed beneath the first lens 440 and the second lens 441 and above the photodetector 403. In this example, the first shutter 410 and the second shutter 411 are configured to selectively block the transmission of the plurality of light signals 430 and 431 to the photodetector 403. In some examples, the first shutter 410 and the second shutter 411 are disposed above the first lens 440 and the second lens 441 respectively and are configured to selectively block the transmission of the plurality of light signals 430 and 431 through the interior volume of the first lens 440 and the second lens 441. In this example, the first shutter 410 and the second shutter 411 are configured to allow the transmission of either the plurality of light signals 430 or 431 according to criteria discussed further below. In some examples, the dual-lens configuration of lens 440 and 441, as illustrated in FIG. 4C, is the same dual-lens configuration described above with reference to FIG. 4A-4B. In some examples, the multidirectional sensor having the dual-lens configuration of lens 440 and 441 illustrated by FIG. 4C includes the photodetector 403 comprising one or more pixels. In some examples, the one or more pixels of photodetector 403 are arranged in a two-dimensional array of pixels in a configuration discussed in further detail below with reference to FIGS. 5A-5E. In some examples, the photodetector 403 receives a plurality of light signals from multiple directions via the first lens 440 and the second lens 441. In some examples, the plurality of light signals includes the plurality of light signals 430 and the plurality of light signals 431 as discussed above in reference to FIGS. 4A-4B. In some examples, the first lens 440 and the second lens 441 include a translucent optical material capable of allowing the transmission of light in the thermal energy spectrum through the volume of either lens. In some examples, operation of the first shutter 410 and the second shutter 411 are time-division multiplexed. For example, as illustrated in FIG. 4C, the second shutter 411 is configured to block the plurality of light signals 431 from the second direction from reaching the photodetector 403 while simultaneously the first shutter 410 is configured to allow the plurality of light signals 430 from the first direction to pass through the interior volume of the first lens 440 to the photodetector 403. In another example, the second shutter 411 is configured to be temporarily opaque via a liquid crystal display and/or a mechanical shutter structure. In accordance with the time-division multiplex of the first shutter 410 and the second shutter 411, the opacity of the first shutter 410 is configured to be translucent for a predetermined time while the second shutter 411 is configured to be opaque, for example. In this example, after the predetermined time, the opacity of each shutter is alternated and held for the predetermined time as further discussed below with reference to FIG. 4D. In some examples, the opacity of the first shutter 410 and the second shutter 411 are both configured to be opaque for the predetermined time in the sequence of altering the opacity of shutters 410 and 411. In some examples, both shutters 410 and 411 are configured to be translucent for the predetermined time in the sequence of altering the opacity of shutters 410 and 411.

FIG. 4D illustrates the first lens 440 and the second lens 441 in the dual-lens configuration possessing the first shutter 410 and the second shutter 411 in a second stage of the time-division multiplexing with reference to FIG. 4C in accordance with some examples of the disclosure. In some examples, the first shutter 410, is configured to be opaque, blocking the transmission of the plurality of light signals 430 from the first direction from passing through the first lens 440 to the photodetector 404. For example, in accordance with a determination by the processing circuitry of a wearable device 301 incorporating the photodetector 404 of FIG. 4D that the predetermined detection time has passed, the shutter system will alternate the opacity of the first shutter and the second shutter, wherein the opacity of the second shutter, being opaque as illustrated in FIG. 4C, is configured to be translucent, and the opacity of the first shutter is configured to be opaque, as illustrated in FIG. 4D. As illustrated in FIGS. 4C-4D, the first shutter 410 and the second shutter 411 alternate between translucent and opaque in a time-division multiplexing fashion according to some examples of the disclosure.

FIG. 4E illustrates an example configuration of a photodetector 405 including shutter rows 461 through 463 with varying levels of opacity controlled by the wearable device 301 in accordance with some examples of the disclosure. In some examples, the photodetector 405 comprises one or more pixels. In some examples, a three-by-three, two-dimensional shutter system, as illustrated in FIG. 4E by shutter rows 461 through 463, is disposed above the photodetector 405. In some examples the photodetector 405 is placed under an optical lens 470 and configured to disperse a plurality of light signals 432 evenly across the photodetector 405. In some examples, the shutter rows 461 through 463 are configured with varying opacities (e.g., 0% opaque, 25% opaque, 50% opaque, 75% opaque, 100% opaque) in a plurality of combinations controlled by the wearable device 301 as discussed further below.

In some examples, the wearable device 301 controls the levels of opacity of the shutter rows 461 through 463, including operating one or more shutter rows in an inactive state and/or one or more shutter rows in an active state. For example, operating one or more shutter rows in the inactive state allows transmission of light signals through those shutter rows. As another example, operating one or more shutter rows in the active state prevents or reduces transmission of light signals through those shutter rows.

For example, shutter row 463 is engaged in the inactive state, as illustrated in FIG. 4E. While shutter row 463 is engaged in the inactive state, shutter row 463 allows the transmission of the plurality of light signals 432 to a subsection of the photodetector 405 corresponding to the shutter row 463. In some examples, the shutter row 463 comprises a liquid crystal display akin to shutter 410 and 411 as discussed above with reference to FIG. 4C. In some examples, the photodetector is disposed within the wearable device 1-106 as discussed above with reference to FIGS. 1A-1B. In some examples, the electronic component 1-112 as discussed with reference FIGS. 1A-1B corresponds to a processor configured to control (e.g., alter) the opacity of the shutter rows 461 through 463. For example, the electronic component 1-112 is configured to set the opacity of shutter row 463 at a value ranging from 0% opaque to 100% opaque. In some examples, the shutter row 463 transitions over a negligible timescale (e.g., 2 ms, 5 ms, 7 ms, 10 ms). In some examples, the opacity of the shutter row 463 is held for a predetermined time dictated by the processor corresponding to the electronic component 1-112, the electronic component 1-112 transitioning the shutter row 463 from a first opacity to a second opacity after the predetermined time has elapsed. For example, the shutter row 463 possesses an opacity level of 0%, allowing the transmission of the plurality of light signals 432 to the photodetector 405. After the predetermined time has elapsed, the shutter row transitions to an opacity level of 100% as discussed in further detail below with reference to FIG. 4F. In some examples, shutter rows 461 and 462 have an opacity level different than the opacity level of shutter row 463, such as a higher level of opacity than shutter row 463. In some examples, the plurality of light signals 432 detected by the subsection of the photodetector 405 corresponding to shutter row 463 are stored for later use by memory 200 (as discussed above with reference to FIG. 2) discussed further below in reference to FIG. 4H.

FIG. 4F illustrates an example configuration of the photodetector 405 including shutter rows 461 through 463 corresponding to the configuration discussed above with reference to FIG. 4E, where the shutter row 462 is configured with an opacity different than the opacities of shutter rows 461 and 463. For example, the shutter rows 461 and 463 are configured by the electronic component 1-112 as discussed above with reference to FIG. 1A to be 100% opaque, thus preventing or significantly reducing the transmission of the plurality of light signals 432 from reaching a subsection of the photodetector 405 corresponding to shutter rows 461 and 463. In this example, the subsection of the photodetector 405 includes one or more pixels of a plurality of pixels of the photodetector 405. In some examples, the opacity of shutter row 462 is configured to be 100% transparent. In some examples, the opacities of shutter rows 461, 462, and 463 are all distinct. In some examples, the photodetector 405 comprises a three-by-three array of pixels with the shutter row 462 overlaying a row of three pixels of the photodetector 405. In some examples, the shutter row 462 is configured to be translucent for the predetermined time as discussed above with reference to FIG. 4E. After the predetermined time, the electronic component 1-112, as discussed above with reference to FIG. 1A, is configured to alter the opacity from the first opacity to the second opacity as discussed further below with reference to FIG. 4G. In some examples, the plurality of light signals 432 detected by the subsection of the photodetector 405 corresponding to shutter row 462 are stored for later use by memory 200 (as discussed above with reference to FIG. 2) discussed further below in reference to FIG. 4H.

FIG. 4G illustrates an example of FIG. 4F, where the shutter row 461 is configured with an opacity less than the opacities of shutter rows 462 and 463 according to some examples of this disclosure. In some examples, the shutter row 461 is configured to be translucent, allowing the transmission of the plurality of light signals 432 to be detected by a subsection of the photodetector 405. In this example, the subsection of the photodetector is different than the subsections of the photodetector exposed by shutter rows 462 and 463. In some examples, the shutter row 461 is configured to be translucent for the predetermined time as discussed above with reference to FIG. 4E. After the predetermined time, the electronic component 1-112, as discussed above with reference to FIG. 1A, is configured to alter the opacity of shutter row 461 from translucent to opaque. In some examples, the shutter row 461 possesses the first opacity. In some examples, the plurality of light signals 432 detected by the subsection of the photodetector 405 corresponding to shutter row 461 are stored for later use by memory 200 (as discussed above with reference to FIG. 2) discussed further below in reference to FIG. 4H.

FIG. 4H illustrates an example of a combined plurality of light signals 480 comprising the plurality of light signals 432 detected by the subsections of the photodetector 405 corresponding to shutter rows 461 through 463 in accordance with some examples of the disclosure. In some examples, the combined plurality of light signals 480 are processed via the processor(s) 218 discussed above with reference to FIG. 2 to generate the breathing signal. In some examples, the combined plurality of light signals 480 are associated with changes in temperature at a user (e.g., the user 300, the user 500, and the user 600), wherein the plurality of light signals 432 are generated by the aforementioned example users. In this example, the combined plurality of light signals 480 can form an image of the user (e.g., the user 300, the user 500, the user 600, see FIG. 5E) possessing multiple (e.g., three) levels of opacity, where the shutter rows 461 through 463 are optionally associated with different respective levels of opacity. In some examples, a lower level of opacity associated with a shutter row corresponds to an area of interest. For example, the shutter rows 461 through 463 may form a two-dimensional matrix configured to mask a subsection of the photodetector 405 where an area of interest corresponding to one or more of the shutter rows 4691 through 463 is masked with a lower level of opacity. In this example, the resulting image detected by the photodetector 405 would include low-interest regions of higher opacity corresponding to one or more of the shutter rows 461 through 463 and high-interest regions of lower opacity corresponding to one or more of the shutter rows 461 through 463. In some examples, the combined plurality of light signals 480 is generated by combining the plurality of light signals 432 detected by the subsections of photodetector 405 discussed above with reference to FIGS. 4E-4G. In this example, the combined plurality of light signals 480 is processed by computer circuitry to generate the breathing signal as discussed further below in reference to FIG. 5E.

FIGS. 5A-5E illustrate examples of an array of pixels 520 through 523 at a multidirectional sensor operating in a time-division multiplexed manner to generate a thermal image of a user 500 in accordance with some examples of this disclosure. In some examples, the array of pixels 520 through 523 includes a two-dimensional array of pixels, where the array of pixels 520 through 523 is a component of a multidirectional sensor included in a wearable device. For example, FIGS. 5A-5E illustrate a two-by-two array of pixels within a multidirectional sensor 502. In some examples, the wearable device 501 and the user 500 correspond to the wearable device 301 and the user 300 as discussed with reference to FIGS. 3A-3D. In some examples, the multidirectional sensor 502 includes two fields of view corresponding to two sensing ranges of the multidirectional sensor 502. For example, a first field of view 510 corresponding to a first sensing range encompasses a first subsection of a target area of the user 500, where the target area includes the first subsection corresponding to the user 500 nose and a second subsection corresponding to the user 500 mouth. In another example, a second field of view 511 corresponding to a second sensing range of the multidirectional sensor encompasses the second subsection of the target area of the user 500. In some examples, the first field of view captures a plurality of light signals corresponding to the plurality of light signals 430, and the second field of view captures a plurality of light signals corresponding to the plurality of light signals 431 as discussed above with reference FIGS. 4A-4D. In some examples, pixels 520 through 523 correspond to photodetector 405 as discussed above with reference to FIGS. 4E-4H. In some examples, pixels 520 through 523 are arranged in the two-dimensional array in a similar fashion to photodetectors 403 and 404 as discussed above with reference to FIGS. 4C-4D. In some examples, the multidirectional sensor 502 includes a dual-shutter configuration corresponding to the first shutter 410 and the second shutter 411 as discussed above with reference to FIG. 4A. In this example, the pixels 520 through 523 are disposed beneath the first shutter 410 and the second shutter 411 and are configured to receive a plurality of light signals from multiple directions (e.g., the plurality of light signals 430 and the plurality of light signals 431) via a three-dimensional configuration of the aforementioned pixels 520 through 523. In some examples, the plurality of light signals detected by pixels 520 through 523 are stored at the multidirectional sensor 502 and processed by computer circuitry to generate the breathing signal discussed in further detail with reference to FIG. 5E.

FIG. 5A illustrates an example configuration of the multidirectional sensor 502 affixed to the wearable device 501 where the multidirectional sensor 502 includes an active pixel 520. In some examples, the pixel 520 corresponds to the first field of view 510 and the second field of view 511. In some examples, the pixel 520 corresponds to one of the fields of view 510 and 511. For example, the pixel 520 detects the plurality of light signals within the field of view 510. In this example, the detected plurality of light signals within the field of view 510 are detected at the multidirectional sensor 502. In some examples, pixel 520 corresponds to the field of views 510 and 511 and include the plurality of light signals 432 discussed above with reference to FIGS. 4E-4H. In some examples, the plurality of light signals are stored at memory of the wearable device to generate a thermal image 530 of the user 500 discussed further below in reference to FIG. 5E. In some examples, the multidirectional sensor 502 deactivates the pixel 520 after the predetermined time discussed above with reference to FIG. 3C. In this example, pixel 523 is activated by the multidirectional sensor 502 and configured to receive the plurality of light signals as discussed further below.

FIG. 5B illustrates the example configuration of the multidirectional sensor 502 described above with reference to FIG. 5A where the active pixel corresponds to pixel 523 according to some examples of this disclosure. In some examples, the pixel 523 is activated in response to the predetermined time elapsing for a detection period in reference to pixel 520. In this example, pixel 523 is activated for the predetermined time and detects the plurality of light signals at the multidirectional sensor 502. In some examples, the pixel 523 corresponds to a field of view different than the field of view detected by the pixel 520. For example, the pixel 520 is activated by the multidirectional sensor 502 and detects the plurality of light signals associated with the field of view 510 during the predetermined time. After the predetermined time, the pixel 520 is deactivated and pixel 523 is activated by the multidirectional sensor 502 and detects the plurality of light signals associated with the field of view 511 during the predetermined time. In some examples, the plurality of light signals detected by the pixel 523 are stored at memory of the wearable device to generate the thermal image 530 associated with the user 500 discussed further below with reference to FIG. 5E. In some examples, the pixel 523 is deactivated after the predetermined time. In this example, the pixel 522 is activated by the multidirectional sensor 502 and configured to receive the plurality of light signals as discussed further below.

FIG. 5C illustrates the example configuration of the multidirectional sensor 502 described above with reference to FIG. 5A where the active pixel corresponds to pixel 522 according to some examples of this disclosure. In some examples, the pixel 522 is activated in response to the predetermined time elapsing for a detection period in reference to pixel 523. In this example, pixel 522 is activated for the predetermined time and detects the plurality of light signals at the multidirectional sensor 502. In some examples, the pixel 522 corresponds to a field of view different than the field of view detected by the pixel 520 and the pixel 523. In some examples, the pixel 522 detects the field of view 510 and/or 511, wherein neither pixel 520 or 523 are configured to detect either field of views referenced. In some examples, the plurality of light signals detected by pixel 520, pixel 522, and pixel 523 and stored at memory of the wearable device in tandem to generate the thermal image 530 associated with the user 500. In this example, the plurality of light signals detected by pixel 520, pixel 522, and pixel 523 correspond to respective subsections of the user 500 face as discussed further below in reference to the thermal image 530. In some examples, the pixel 522 is deactivated after the predetermined time. In this example, the pixel 521 is activated by the multidirectional sensor 502 and configured to receive the plurality of light signals as discussed further below.

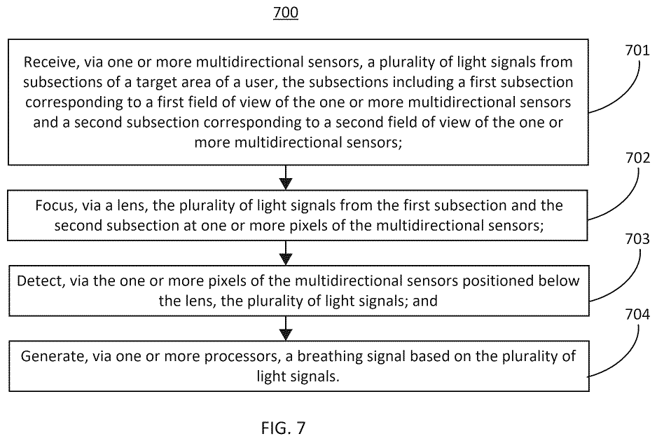

FIG. 5D illustrates the example configuration of the multidirectional sensor 502 described above with reference to FIG. 5A where the active pixel corresponds to pixel 521 according to some examples of this disclosure. In some examples, the pixel 521 is activated in response to the predetermined time elapsing for a detection period in reference to pixel 522. In this example, pixel 521 is activated for the predetermined time and detects the plurality of light signals at the multidirectional sensor 502. In some examples, the pixel 521 corresponds to a field of view different than the field of view detected by the pixel 520, the pixel 523, and/or the pixel 522. In some examples, the pixel 521 detects the field of view 510 and/or 511, wherein neither pixel 520, 522, or 523 are configured to detect either field of views referenced. In some examples, the plurality of light signals detected by pixel 520, pixel 522, pixel 523, and pixel 521 and stored at memory of the wearable device in tandem to generate the thermal image 530 associated with the user 500. In this example, the plurality of light signals detected by pixel 520, pixel 522, pixel 523, and pixel 521 each correspond to a subsection of the user 500 face as discussed further below in reference to the thermal image 530. In some examples, the pixel 521 is deactivated after the predetermined time. In this example, the deactivation of pixel 521 triggers a processing step discussed below with reference to block 704 of the method 700 illustrated by FIG. 7 below.

FIG. 5E illustrates an example of the thermal image 530 of the user 500 via the detected plurality of light signals by pixels 520 through 523 according to some examples of this disclosure. In some examples, the thermal image 530 illustrates changes in temperature at a target area of the user 500 (e.g., the light signals generated by changes in temperature at the user 300 detected within the field of view 310 as discussed above with reference to FIG. 3A). In some examples, the changes in temperature at the user 500 are induced by breathing. For example, the user 500 exhales for a period of time (e.g., 0.5 seconds, 1 second, 1.5 seconds, or 2 seconds), resulting in the warming of the skin of the user 500. In this example, the warming of the skin results in a higher temperature at the nose and mouth of the user when compared to the ambient temperature of the environment represented by the darker shading of area 535 and 532 as illustrated by FIG. 5E. In some examples, area 535 corresponds to the field of view 510 and area 532 corresponds to the field of view 511 as discussed above with reference to FIGS. 5A-5D. In some examples, pixels 520 through 523 detect the changes in temperature of an area of the user 500. For example, pixel 520 detects a plurality of light signals at area 533, pixel 521 detects a plurality of light signals at area 534, pixel 522 detects a plurality of light signals area 535, and pixel 523 detects a plurality of light signals at area 532 and 531. In this example, each of the pixels 520 through 523 detects and stores the plurality of light signals during the predetermined time as discussed above with reference to FIG. 3C. In some examples, the detection of areas 531 through 535 by pixels 520 through 523 are staggered. For example, pixel 520 detects area 535 for a first period of time, wherein the multidirectional sensor 502 deactivates pixel 520 and activates pixel 521 to detect area 532 for a second period of time. In some examples, the first period of time and the second period of time are equal. In some examples, the first period of time and the second period of time are different. In some examples, the pixels 520 through 523 are active and detecting all areas of thermal image 530 during the predetermined time discussed above with reference to FIG. 3C. In some examples, pixels 520 through 523 are time-division multiplexed to form the thermal image 530. In some examples, the changes in temperature illustrated by the darker regions of thermal image 530 are induced by the user 500 inhaling, resulting in a drop of temperature at the skin of user 500. In some examples, the thermal image 530 corresponds to the breathing signal as discussed above with reference to FIG. 3A. In this example, a first plurality of light signals (e.g., sₐ(t)) are detected at any of the pixels 520 through 523 during a first detection phase and a second plurality of light signals (e.g., s_{b}(t)) are detected at any of the pixels 520 through 523 during a second detection phase. Using the computer circuitry at multidirectional sensor 502 (e.g., processor(s) 218), sₐ(t) and s_{b}(t) are each assigned a weight to generate the breathing signal (e.g., s(t)=wₐsₐ(t−φ)+ws_{b}t)) associated with the user 500, where φ is a phase shift. The weights and the phase shift are selected to increase the signal-to-noise ratio of the derived breathing signal.

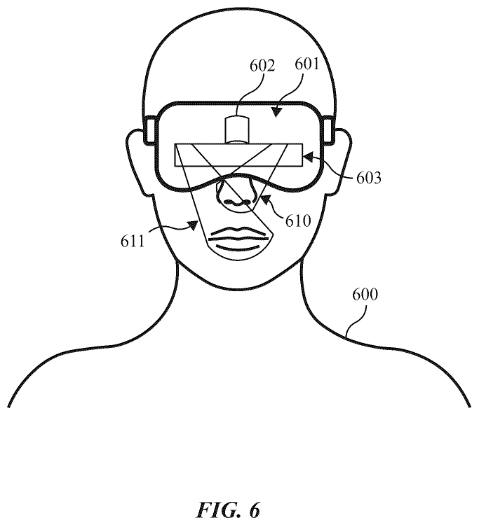

FIG. 6 illustrates an example of wearable device 601 including a multidirectional sensor 602 that includes an optical waveguide 603 in accordance with some examples of the disclosure. In some examples, the optical waveguide 603 directs light from a first field of view 610 and a second field of view 611. In some examples, the first field of view 610 corresponds to a first subsection of a target area of the user 600 face, and the second field of view 610 corresponds to a second subsection of a target rea of the user 600 face. For example, the optical waveguide 603 is configured to channel light from the first subsection of the user 600 in the first field of view 610 to a first end of the optical waveguide 603 and channel light from the second subsection of the user 600 in the second field of view 611 to a second end of the optical waveguide 603. In some examples, the first end and the second end of the optical waveguide 603 act as receivers for light within the first field of view 611 and the second field of view 610 and transmit the respective light along the interior volume of the optical waveguide 603 to be received at the multidirectional sensor 602. In some examples, the optical waveguide 603 possesses a core structure that incorporates a refractive index profile, enabling the confinement of light from the first field of view 610 and the second field of view 611 and their subsequent propagation through the interior volume of the optical waveguide 603. In some examples, surrounding the core are cladding layers, each possessing a distinct refractive index to confine the light within the core and prevent leakage. The cladding layers may include materials like polymers, oxides, or other dielectrics, strategically chosen to optimize the performance of the optical waveguide 603. In some examples, the optical waveguide 603 is a planar structure possessing a flat distal end and a flat proximal end, wherein the distal end and the proximal end act as receivers for the plurality of light signals of the first field of view 610 and the second field of view 611 respectively. In additional examples, the optical waveguide 603 is curved in accordance with the curvature of the display assembly 1-108 discussed above with reference to FIGS. 1A-1B. In some examples, the optical waveguide 603 is disposed within or outside the wearable device 601 and is configured to attach perpendicular to the multidirectional sensor 602. In some examples, the multidirectional sensor 602 possesses a single field of view, where the optical waveguide 603 channels the light from the first field of view 610 and the second field of view 611 to be detected within the field of view of the multidirectional sensor 602. In some examples, the wearable device 601 and the user 600 correspond to the wearable device 301 and the user 300 as discussed above with reference to FIGS. 3A-3D. In some examples, the light from the first field of view 610 and the light from the second field of view 611 are time-division multiplexed at the multidirectional sensor 602 in a similar fashion as discussed above with reference to the FIG. 3, FIG. 4, and FIG. 5 series.

FIG. 7 is a flow diagram illustrating a method 700 of generating a breathing signal associated with the user 300 according to some examples of this disclosure. The method is optionally performed at a wearable device as described above with reference to FIGS. 1-2 (e.g., head-mounted display 1-100, or device 201). Some operations in method 700 are, optionally, combined and/or the order of some operations is, optionally, changed. In some examples, the method 700 comprises four steps (e.g., blocks 701 through 704).

In some examples, block 701, in accordance with the method 700, involves receiving of a plurality of light signals emanating from subsections within a designated target area of a user via a multidirectional sensor according to some examples of this disclosure. In some examples, the plurality of light signals corresponds to the plurality of light signals 430 and 431 with reference to FIGS. 4A-4D as discussed above. In some examples, the detection step is facilitated through the utilization of one or more multidirectional sensors optimally positioned to capture biometric data from the user. For example, the one or more multidirectional sensors correspond to sensors 303 and 304 as discussed above with reference to FIG. 3B. In some examples, the one or more multidirectional sensors comprise only a single multidirectional sensor corresponding to either multidirectional sensor 305 or 306 as discussed above with reference to FIGS. 3C-3D. In some examples, a single multidirectional sensor such as multidirectional sensor 305, includes the target area, where the target area is segmented into subsections (e.g., the field of view 310 and 311 as illustrated in FIG. 3B), each serving to detect light from a certain area of the multidirectional sensor's field of view. In some examples, the first subsection corresponds to the first field of view 610 of the multidirectional sensor illustrated in FIG. 6, while the second subsection aligns with the second field of view 611 of the same sensor. This systematic capture of the plurality of light signals from various directions from these subsections sets the foundation for subsequent processing steps of method 700.

In some examples, block 702, in accordance with method 700, involves focusing the plurality of light signals from the first subsection and the second subsection of the target area, via a lens according to some examples of this disclosure. In some examples, the lens of block 702 corresponds to the optical lens of sensor 302 as discussed above with reference to FIG. 3A and/or the optical lens 470 as discussed above with reference to FIG. 4E. In some examples, the lens is a dual-lens configuration corresponding to the first lens 340 and the second lens 341 as discussed above with reference to FIG. 3E. In this example, the dual-lens configuration previously discussed focuses the plurality of light signals in reference to block 702 from multiple directions (e.g., the first subsection and the second subsection) onto one or more pixels. In some examples, the lens focuses the plurality of light signals onto a photodetector corresponding to the photodetector 405 as discussed above with reference to FIG. 4E. In some examples, the aforementioned photodetector includes one or more pixels configured to detect the plurality of light signals as discussed in further detail below with reference to block 703. In some examples, the one or more pixels of block 702 are arranged in any of the configurations corresponding to the multidirectional sensors 302 through 306 discussed above with reference to FIGS. 3A-3D.

In some examples, at block 703, in accordance with method 700, the one or more pixels of the photodetector as discussed above with reference to block 702 detects the focused plurality of light signals. In some examples, the detection of the plurality of light signals from the first subsection and the second subsection of the target area of block 702 are staggered. For example, the plurality of light signals from the first subsection of the target area are detected by the one or more pixels for the predetermined time discussed above with reference to FIG. 3C, where after the predetermined time, the plurality of light signals from the second subsection of the target area are detected by the one or more pixels for the predetermined time. In some examples, the one or more pixels of the multidirectional sensor are positioned below the lens with reference to block 702. For example, the lens and the multidirectional sensor are configured in a similar fashion to the first lens 340, the second lens 341, and the pixel 320 as discussed above with reference to FIG. 3E. In some examples, the detected plurality of light signals at the one or more pixels of the multidirectional sensor are converted to a plurality of electronic signals and stored by computer circuitry at the multidirectional sensor for generating a breathing signal as discussed further below with reference to block 704.

In some examples, at block 704, in accordance with method 700, the plurality of electronic signals associated with the plurality of breathing signals are processed to generate a breathing signal associated with the user of the multidirectional sensor. In some examples, the plurality of light signals are restricted by the lens in reference to block 702 to include the total plurality of light signals produced by the user and without including light signals produced by an environment the multidirectional sensor is within. In this example, the plurality of light signals produced by the user are one source of light signals amongst the environment the multidirectional sensor is within, and in restricting the multidirectional user to detect the plurality of light signals produced by the user, via the lens in reference to block 702, extraneous signals are removed or reduced during the processing stage of the plurality of light signals. In some examples, the generation of the breathing signal is a continuous process, wherein a breathing signal is generated that is associated with the plurality of light signals detected during the predetermined time in reference to block 703. In some examples, the breathing signal is generated via the weighted combination and/or phase-shifts of the first plurality of light signals and the second plurality of light signals as discussed above with reference to FIG. 5E.

It should be understood that the particular order in which the blocks of the flowchart of FIG. 7 have been described is merely exemplary and is not intended to indicate that the described order is the only order in which the operations could be performed. One of ordinary skill in the art would recognize various ways to reorder the operations described herein.

Therefore, according to the above, some examples of the disclosure are directed to a wearable device comprising: one or more one or more multidirectional sensors configured to receive a plurality of light signals from subsections of a target area of a user, the subsections including a first subsection corresponding to a first field of view of the one or more multidirectional sensors and a second subsection corresponding to a second field of view of the one or more multidirectional sensors; a lens configured to focus the plurality of light signals received from the first subsection and the second subsection; one or more pixels positioned below the lens configured to detect the plurality of light signals received from the first subsection and the second subsection; and one or more processors configured to: generate a breathing signal based on the plurality of light signals. Additionally or alternatively to one of more of the examples disclosed above, in some examples, the lens is a dual-shaped lens comprising: a first lens positioned above the one or more pixels, and a second lens positioned above the one or more pixels. The first lens is angled towards the first subsection, the first lens and the second lens are arranged in parallel above the one or more pixels, and the second lens is angled towards the second subsection. Additionally or alternatively to one of more of the examples disclosed above, in some examples, the plurality of light signals from the first subsection are associated with a change in temperature at a first location at the user of the wearable device. The plurality of light signals from the second subsection are associated with a change in temperature at a second location, different than the first location, at the user. Additionally or alternatively to one of more of the examples disclosed above, in some examples, the wearable device further comprising a shutter system disposed within an interior volume of the lens, the shutter system including: a first shutter configured with an active state and an inactive state and a second shutter, additionally configured with the active state and the inactive state, positioned in the interior volume of the second lens. the active state of the first shutter blocks transmission of the plurality of light signals from the first subsection through an interior volume of the first lens to be detected by the one or more pixels. The inactive state of the first shutter allows a transmission of the plurality of light signals from the second subsection through the interior volume of the second lens to be detected by the one or more pixels. The active state of the second shutter blocks the transmission of the plurality of light signals from the first subsection through an interior volume of the second lens to be absorbed by the one or more pixels, and the inactive state of the second shutter allows the transmission of the plurality of light signals from the second subsection through the interior volume of the second lens to be detected by the one or more pixels. Additionally or alternatively to one of more of the examples disclosed above, in some examples, the first shutter and the second shutter include a silicon cover window. Additionally or alternatively to one of more of the examples disclosed above, in some examples, the first lens and the second lens of the shutter system includes a liquid crystal display; the one or more processors are configured to time division multiplex the first lens and the second lens; in accordance with a determination that the first lens is in the active state, configure the liquid crystal display of the first lens with a first opacity and configure the second lens to be in the inactive state, wherein the liquid crystal display of the second lens is configured with a second opacity that is less than the first opacity; and in accordance with a determination that the first lens is in the inactive state, configure the liquid crystal display of the first lens with the second opacity and configure the second lens to be in the active state, wherein the liquid crystal display of the second lens is configured with the first opacity. Additionally or alternatively to one of more of the examples disclosed above, in some examples, the one or more processors are additionally configured to time division multiplex the shutter system. The shutter system is further configured to: in response to receiving an activation signal from the one or more processors: activate, a first subset of the one or more pixels; and deactivate, via a deactivation signal from the one or more processors, a second subset of the one or more pixels. Additionally or alternatively to one of more of the examples disclosed above, in some examples, the one or more pixels comprises a two-dimensional array of pixels configured with the active state and the inactive state; and the one or more processors are further configured to: during a first time, configure the pixels so that a first pixel has the active state and a second set of the one or more pixels has the inactive state in accordance with the time division multiplex of the shutter system; and during a second time different from the first time, configure the pixels so that the second of the one or more pixels has the active state and the first set of one or more pixels has the inactive state in accordance with the time division multiplex of the shutter system. Additionally or alternatively to one of more of the examples disclosed above, in some examples, the one or more multidirectional sensors are infrared sensors further comprise: a first pixel oriented towards the first subsection; and a second pixel oriented towards the second subsection. The first pixel focuses the plurality of light signals from the first subsection simultaneously with the second pixel focusing the plurality of light signals from the second subsection. Additionally or alternatively to one of more of the examples disclosed above, in some examples, the one or more multidirectional sensors further comprising: a single-pixel triangular configuration including a first side and a second side. The first side is positioned perpendicular to the plurality of light signals from the first subsection, the second side is positioned perpendicular to the plurality of light signals from the second subsection, and the first side and the second side receive their respective plurality of light signals simultaneously. Additionally or alternatively to one of more of the examples disclosed above, in some examples, the plurality of light signals from the first subsection and the plurality of light signals from the second subsection are infrared radiation. Additionally or alternatively to one of more of the examples disclosed above, in some examples, the wearable device includes an optical waveguide body comprising: a first receiving end, the first receiving end configured to receive the plurality of light signals from the first subsection; a central emitter; a second receiving end, the second receiving end configured to receive the plurality of light signals from the second subsection; and a central pathway disposed along the first receiving end and the second receiving end, the central pathway configured to: transmit, via the optical waveguide body, the plurality of light signals from the first subsection to the central emitter along the central pathway; transmit, via the optical waveguide body, the plurality of light signals from the second subsection to the central emitter along the central pathway; and transmit, via the central emitter, the plurality of light signals from the first subsection and the second subsection to the one or more multidirectional sensors.

Some examples of the disclosure are directed to a method for determining a breathing signal of a user comprising: receiving, via one or more multidirectional sensors, a plurality of light signals from subsections of a target area of a user, the subsections including a first subsection corresponding to a first field of view of the one or more multidirectional sensors and a second subsection corresponding to a second field of view of the one or more multidirectional sensors; focusing, via a lens, the plurality of light signals from the first subsection and the second subsection at one or more pixels of the multidirectional sensors; detecting, via the one or more pixels of the multidirectional sensors positioned below the lens, the plurality of light signals; and generating, via one or more processors, a breathing signal based on the plurality of light signals. Additionally or alternatively to one of more of the examples disclosed above, in some examples, the breathing signal is used to determine a cardiac signal associated with the user. Additionally or alternatively to one of more of the examples disclosed above, in some examples, the method further comprises: optimizing, via a feedback loop, the detection of the plurality of light signals; and generating, via the one or more processors, an optimized measurement of the plurality of light signals; and converting, via the one or more processors, the optimized measurement of the plurality of light signals into an optimizing breathing signal associated with the user. Additionally or alternatively to one of more of the examples disclosed above, in some examples, while generating the breathing signal based on the plurality of light signals, assign, via the one or more processors, a first weight to a plurality of light signals from the first subsection and a second weight to a plurality of light signals from the second subsection, and multiply, via the one or more processors the second weight with the plurality of light signals from the second subsection to produce a weighted plurality of light signals from the subsection, and add, via the one or more processors, the weighted plurality of light signals from the first subsection and the weighted plurality of light signals from the second subsection, wherein the weighted plurality of light signals from the first subsection includes a phase shift associated with a detection phase

Some examples of the disclosure are directed to a non-transitory computer readable storage medium storing one or more programs, the one or more programs comprising instructions, which when executed by one or more processors of an electronic device, cause the electronic device to perform a method comprising: receiving, via one or more multidirectional sensors, a plurality of light signals from subsections of a target area of a user, the subsections including a first subsection corresponding to a first field of view of the one or more multidirectional sensors and a second subsection corresponding to a second field of view of the one or more multidirectional sensors; focusing, via a lens, the plurality of light signals from the first subsection and the second subsection at one or more pixels of the multidirectional sensors; detecting, via the one or more pixels of the multidirectional sensors positioned below the lens, the plurality of light signals; and generating, via one or more processors, a breathing signal based on the plurality of light signals.

Although the disclosed examples have been fully described with reference to the accompanying drawings, it is to be noted that various changes and modifications will become apparent to those skilled in the art. Such changes and modifications are to be understood as being included within the scope of the disclosed examples as defined by the appended claims.

1. A wearable device comprising:
one or more multidirectional sensors configured to receive a plurality of light signals from subsections of a target area of a user, the subsections including a first subsection corresponding to a first field of view of the one or more multidirectional sensors and a second subsection corresponding to a second field of view of the one or more multidirectional sensors;
a lens configured to focus the plurality of light signals received from the first subsection and the second subsection;
one or more pixels positioned below the lens configured to detect the plurality of light signals received from the first subsection and the second subsection; and
one or more processors configured to:
generate a breathing signal based on the plurality of light signals.

2. The wearable device of claim 1, wherein the lens is a dual-shaped lens comprising:
a first lens positioned above the one or more pixels, wherein the first lens is angled towards the first subsection; and
a second lens positioned above the one or more pixels, wherein the first lens and the second lens are arranged in parallel above the one or more pixels, wherein the second lens is angled towards the second subsection.

3. The wearable device of claim 1, wherein the plurality of light signals from the first subsection are associated with a change in temperature at a first location at the user of the wearable device; and
wherein the plurality of light signals from the second subsection are associated with a change in temperature at a second location, different than the first location, at the user.

4. The wearable device of claim 2, further comprising a shutter system disposed within an interior volume of the lens, the shutter system including:
a first shutter configured with an active state and an inactive state, wherein the active state of the first shutter blocks transmission of the plurality of light signals from the first subsection through an interior volume of the first lens to be detected by the one or more pixels, and wherein the inactive state of the first shutter allows a transmission of the plurality of light signals from the second subsection through the interior volume of the second lens to be detected by the one or more pixels; and
a second shutter positioned in the interior volume of the second lens, wherein the second shutter is additionally configured with the active state and the inactive state, wherein the active state of the second shutter blocks the transmission of the plurality of light signals from the first subsection through an interior volume of the second lens to be absorbed by the one or more pixels, and wherein the inactive state of the second shutter allows the transmission of the plurality of light signals from the second subsection through the interior volume of the second lens to be detected by the one or more pixels.

5. The wearable device of claim 4, wherein the first shutter and the second shutter include a silicon cover window.

6. The wearable device of claim 5, wherein the first lens and the second lens of the shutter system includes a liquid crystal display;
the one or more processors are configured to time division multiplex the first lens and the second lens;
in accordance with a determination that the first lens is in the active state, configure the liquid crystal display of the first lens with a first opacity and configure the second lens to be in the inactive state, wherein the liquid crystal display of the second lens is configured with a second opacity that is less than the first opacity; and
in accordance with a determination that the first lens is in the inactive state, configure the liquid crystal display of the first lens with the second opacity and configure the second lens to be in the active state, wherein the liquid crystal display of the second lens is configured with the first opacity.

7. The wearable device of claim 5, wherein the one or more processors are additionally configured to time division multiplex the shutter system, wherein the shutter system is further configured to:
in response to receiving an activation signal from the one or more processors:
activate, a first subset of the one or more pixels; and
deactivate, via a deactivation signal from the one or more processors, a second subset of the one or more pixels.

8. The wearable device of claim 7, wherein the one or more pixels comprises a two-dimensional array of pixels configured with the active state and the inactive state; and the one or more processors are further configured to:
during a first time, configure the pixels so that a first set of the one or more pixels has the active state and a second set of the one or more pixels has the inactive state in accordance with the time division multiplex of the shutter system; and
during a second time different from the first time, configure the pixels so that the second set of one more pixels has the active state and the first set of one or more pixels has the inactive state in accordance with the time division multiplex of the shutter system.

9. The wearable device of claim 1, wherein the one or more multidirectional sensors are infrared sensors further comprising:
a first pixel oriented towards the first subsection; and
a second pixel oriented towards the second subsection, wherein the first pixel focuses the plurality of light signals from the first subsection simultaneously with the second pixel focusing the plurality of light signals from the second subsection.

10. The wearable device of claim 1, wherein the one or more multidirectional sensors further comprises:
a single-pixel triangular configuration including a first side and a second side, wherein the first side is positioned perpendicular to the plurality of light signals from the first subsection, wherein the second side is positioned perpendicular to the plurality of light signals from the second subsection; and
wherein the first side and the second side receive their respective plurality of light signals simultaneously.

11. The wearable device of claim 1, wherein the plurality of light signals from the first subsection and the plurality of light signals from the second subsection are infrared radiation.

12. The wearable device of claim 1, wherein the wearable device includes an optical waveguide body comprising:
a first receiving end, wherein the first receiving end is configured to receive the plurality of light signals from the first subsection;
a central emitter;
a second receiving end, wherein the second receiving end is configured to receive the plurality of light signals from the second subsection; and
a central pathway disposed along the first receiving end and the second receiving end, the central pathway configured to:
transmit, via the optical waveguide body, the plurality of light signals from the first subsection to the central emitter along the central pathway;
transmit, via the optical waveguide body, the plurality of light signals from the second subsection to the central emitter along the central pathway; and
transmit, via the central emitter, the plurality of light signals from the first subsection and the second subsection to the one or more multidirectional sensors.

13. A method for determining a breathing signal of a user comprising:
receiving, via one or more multidirectional sensors, a plurality of light signals from subsections of a target area of a user, the subsections including a first subsection corresponding to a first field of view of the one or more multidirectional sensors and a second subsection corresponding to a second field of view of the one or more multidirectional sensors;
focusing, via a lens, the plurality of light signals from the first subsection and the second subsection at one or more pixels of the multidirectional sensors;
detecting, via the one or more pixels of the multidirectional sensors positioned below the lens, the plurality of light signals; and
generating, via one or more processors, a breathing signal based on the plurality of light signals.

14. The method of claim 13, wherein the breathing signal is used to determine a cardiac signal associated with the user.

15. The method of claim 13, further comprising:
optimizing, via a feedback loop, the detection of the plurality of light signals; and
generating, via the one or more processors, an optimized measurement of the plurality of light signals; and
converting, via the one or more processors, the optimized measurement of the plurality of light signals into an optimizing breathing signal associated with the user.

16. The method of claim 13, further comprising:
while generating the breathing signal based on the plurality of light signals:
assigning, via the one or more processors, a first weight to a plurality of light signals from the first subsection and a second weight to a plurality of light signals from the second subsection;
multiplying, via the one or more processors, the first weight with the plurality of light signals from the first subsection to produce a weighted plurality of light signals from the first subsection;
multiplying, via the one or more processors, the second weight with the plurality of light signals from the second subsection to produce a weighted plurality of light signals from the second subsection; and
adding, via the one or more processors, the weighted plurality of light signals from the first subsection and the weighted plurality of light signals from the second subsection, wherein the weighted plurality of light signals from the first subsection includes a phase shift associated with a detection phase.

17. A non-transitory computer readable storage medium storing one or more programs, the one or more programs comprising instructions, which when executed by one or more processors of an electronic device, cause the electronic device to perform a method comprising:
receiving, via one or more multidirectional sensors, a plurality of light signals from subsections of a target area of a user, the subsections including a first subsection corresponding to a first field of view of the one or more multidirectional sensors and a second subsection corresponding to a second field of view of the one or more multidirectional sensors;
focusing, via a lens, the plurality of light signals from the first subsection and the second subsection at one or more pixels of the multidirectional sensors;
detecting, via the one or more pixels of the multidirectional sensors positioned below the lens, the plurality of light signals; and
generating, via one or more processors, a breathing signal based on the plurality of light signals.
